(19) United States
(12) Patent Application Publication
(10) Pub. No.: US 2026/0294237 A1
(43) Pub. Date: Oct. 1, 2026

(54) OPHTHALMIC APPARATUS FOR MULTIPLE EYE HEALTH MEASUREMENTS

(71) Applicant: Hong Kong Applied Science and Technology Research Institute Company Limited, Hong Kong (HK)

(72) Inventors: Pingping FENG, Hong Kong (HK); Ruipeng XU, Shenzhen (CN); Caiyun MIAO, Hong Kong (HK)

(21) Appl. No.: 19/095,083

(22) Filed: Mar. 31, 2025

(51) Int. Cl. A61B 3/107; A61B 3/00; A61B 3/12

(52) U.S. Cl. CPC A61B 3/107; A61B 3/0008; A61B 3/0091; A61B 3/12

(57) ABSTRACT

The present invention provides an ophthalmic apparatus for multiple eye health measurements. The ophthalmic apparatus comprises: an objective lens module configured for magnifying images of a subject's eye; a fixture target configured for stabilizing the subject's eye to a fixation position; a corneal topography measurement unit for measuring geometric parameters of a cornea; a fundus imaging unit for imaging a fundus; a first beam splitter configured for separating or combining optical paths among the objective lens module, the corneal topography measuring unit and the fundus imaging unit; and a second beam splitter configured for diverting an optical beam from the fixture target to the object's eye. The objective lens module, the fixture target, the first beam splitter and the second beam splitter are shared by the corneal topography measurement unit, the fundus imaging unit so that the size and complexity of the overall system can be reduced.

### FIELD OF THE INVENTION

The present invention generally relates to ophthalmic measurement technologies. More specifically the present invention relates to an ophthalmic apparatus capable of performing corneal topography measurement, fundus imaging and axial length estimation.

### BACKGROUND OF THE INVENTION

Eye health and vision play a vital role in overall well-being, yet many people face considerable challenges in accessing affordable, high-quality eye care. These barriers contribute to widespread vision impairment and blindness, despite nearly 50% of such cases being preventable with early diagnosis and regular monitoring. While technological advancements have improved eye care, traditional devices often come with significant drawbacks. Many are designed for a single function, limiting their utility for large-scale screenings. Comprehensive eye exams frequently require multiple devices, leading to prohibitive costs. Additionally, some devices demand manual alignment, which can be cumbersome and time-intensive for less experienced staff.

### SUMMARY OF THE INVENTION

It is an objective of the present invention to provide a single device that allows users to examine multiple parts of the eye to obtain comprehensive corneal, fundus, and axial length information. This innovation would simplify the process of eye health assessments, enhance measurement efficiency, and lower overall costs associated with eye care.

In accordance with a first aspect of the present invention, an ophthalmic apparatus is provided. The apparatus comprises: an objective lens module configured for magnifying images of a subject's eye; a fixture target configured for stabilizing the subject's eye to a fixation position; a corneal topography measurement unit for measuring geometric parameters of a cornea of the subject's eye; a fundus imaging unit for imaging a fundus of the subject's eye; a controller configured for controlling the corneal topography measurement unit and the fundus imaging unit to acquire ophthalmic data of the subject's eye; a processor configured for performing predetermined processing on the acquired ophthalmic data; a first beam splitter configured for separating optical paths from the objective lens module to the corneal topography measuring unit and the fundus imaging unit or combining optical paths to the objective lens module from the corneal topography measuring unit and the fundus imaging unit respectively; and a second beam splitter configured for diverting an optical beam from the fixture target to the object's eye. The objective lens module, the fixture target, the first beam splitter and the second beam splitter are shared by the corneal topography measurement unit, the fundus imaging unit.

### BRIEF DESCRIPTION OF THE DRAWINGS

Embodiments of the invention are described in more details hereinafter with reference to the drawings, in which:

FIGS. 1A and 1B show isometric and side views of an ophthalmic apparatus in accordance with one embodiment of the present invention; FIG. 1C is a simplified schematic diagram for illustrating optical components of the ophthalmic apparatus of FIGS. 1A and 1B;

FIG. 2 shows a schematic diagram for the corneal topography measurement unit in accordance with one embodiment of the present invention;

FIG. 3 shows a ray-tracing model for the corneal topography measurement unit of FIG. 2;

FIG. 4A shows a Placido ring light generator in accordance with one embodiment of the present invention; and FIG. 4B shows a Placido disk formed by the Placido ring light generator.

FIG. 5 shows a ray tracing of a LED emitter in accordance with one embodiment of the present invention;

FIGS. 6A to 6D show front isometric view, rear isometric views, front view and rear view of a primary lens of the LED emitter;

FIGS. 7 and 8 show beam profile measurement results of the LED emitter;

FIG. 9 shows a schematic diagram for the fundus imaging unit in accordance with one embodiment of the present invention;

FIGS. 10A and 10B show ray-tracing models for an illumination optical path and an imaging optical path of the fundus imaging unit, respectively;

FIG. 11 shows an exemplary design for a fundus illumination light source;

FIGS. 12A and 12B show front view and rear view of the illumination light source respectively;

FIG. 13 shows an exemplary design for a gradient light blocking filter;

FIGS. 14A and 14B show simulation of fundus image obtained with the fundus imaging unit in accordance with the present invention and a comparative design which using a single LED without stray light elimination unit;

FIG. 15 shows a corneal image obtained by the ophthalmic apparatus provided by the present invention;

FIGS. 16A and 16B show 3D models of corneal curvature and corneal height reconstructed from the corneal image data respectively; and

FIG. 17 shows a fundus image obtained by the ophthalmic apparatus provided by the present invention.

### DETAILED DESCRIPTION

In the following description, details of the present invention are set forth as preferred embodiments. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

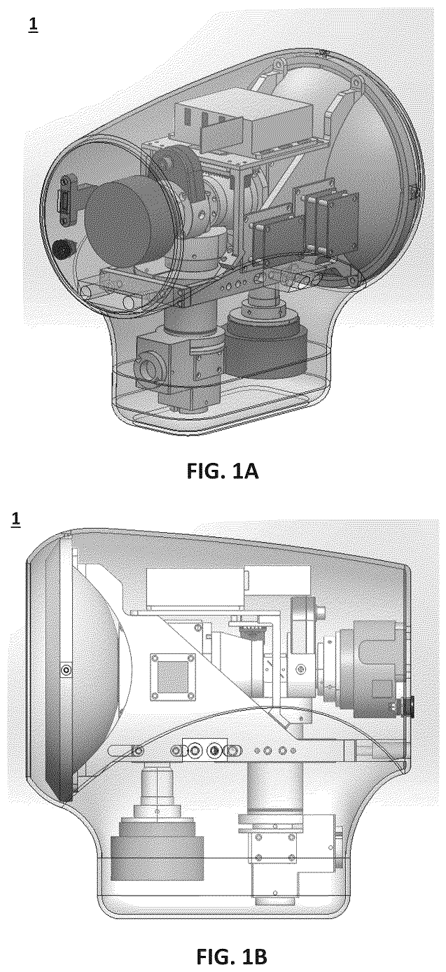

FIGS. 1A and 1B illustrate an ophthalmic apparatus 1 in accordance with one embodiment of the present invention; and FIG. 1C is a simplified schematic diagram showing optical components of the ophthalmic apparatus. As shown, the ophthalmic apparatus 1 comprises a corneal topography measurement unit 2 for measuring geometric parameters of a cornea of a subject's eye E and a fundus imaging unit 3 for imaging a fundus of the subject's eye.

The ophthalmic apparatus 1 comprises an objective lens module 11 configured for light collection and initial focusing of a subject's eye. The objective lens module 11 may include one or more lenses sequentially aligned along an optical axis of the objective lens module 11.

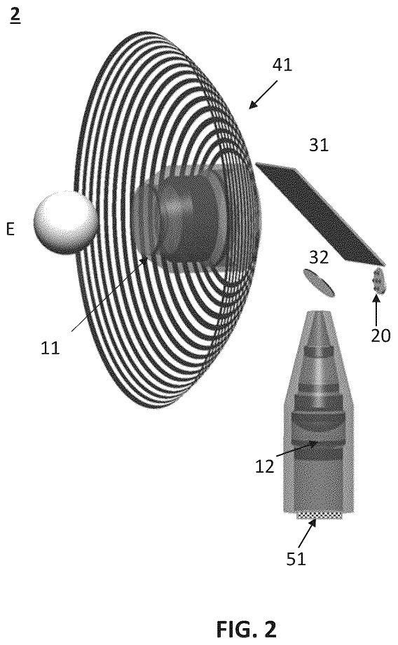

Referring to FIG. 3, in one embodiment, the objective lens module 11 may include a first lens 111, a second lens 112 positioned next to the first lens 111, a third lens 113 positioned next to the second lens 112 and a fourth lens 114 positioned next to the third lens 113. The first lens 111 may be a plano-convex lens. The second lens 112 may be a biconvex lens. The third lens 113 may be a biconcave lens. The second and the third lenses 112 and 113 may form a doublet lens. The fourth lens 114 may be a biconvex lens. Each of the lenses 111 to 114 may made of glass, plastics or any suitable optical materials.

The ophthalmic apparatus 1 further comprises a fixture target 20 configured for stabilizing the subject's eye to a fixation position. The fixture target 20 may include a plurality of light-emitting diode (LEDs) arranged in any suitable patterns. For example, the fixture target 20 may include a first LED positioned at a central spot and multiple second LEDs arranged in a circular and symmetrical pattern around the first LED.

The ophthalmic apparatus 1 further comprises a first beam splitter 31 configured for separating optical paths from the objective lens module 11 to the corneal topography measuring unit and the fundus imaging unit 3 or combining optical paths to the objective lens module 11 from the corneal topography measuring unit and the fundus imaging unit 3 respectively.

The ophthalmic apparatus 1 further comprises a second beam splitter 32 configured for diverting an optical beam from the fixture target 20 to the object's eye.

The ophthalmic apparatus 1 further comprises a controlling unit (not shown) configured for controlling the corneal topography measurement unit 2 and the fundus imaging unit 3 to acquire ophthalmic data of the subject's eye; and a processing unit (not shown) configured for performing present processing on the acquired ophthalmic data. The ophthalmic apparatus 1 may further comprise a storage unit (not shown) for storing the acquired data and preset processing methods and algorithms. Alternatively, the controlling unit and the processing unit may include respective storage memory for storing the acquired data and preset processing methods and algorithms.

The objective lens module 11, the fixture target 20, the first beam splitter 31 and the second beam splitter 32 are shared by the corneal topography measurement unit 2 and the fundus imaging unit 3 so that the size and complexity of the overall system can be reduced.

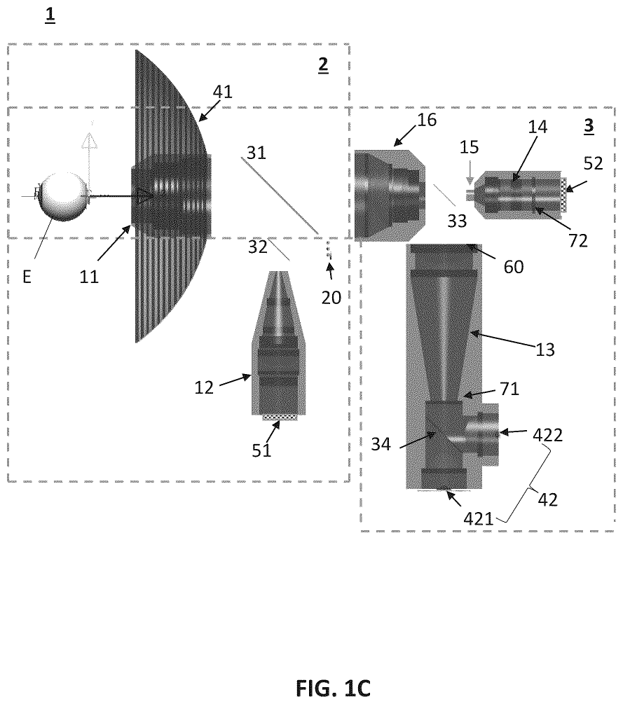

FIG. 2 shows a schematic diagram for the corneal topography measurement unit 2; and FIG. 3 shows an exemplary ray-tracing model for the corneal topography measurement unit 2.

As shown, in addition to the objective lens module 11, the fixture target 20, the first beam splitter 31 and the second beam splitter 32, the corneal topography measurement unit 2 further includes a projector 41 configured to project a ring light pattern onto the cornea of the subject's eye; a corneal image sensor 51 configured to detect returning light of the ring light pattern reflected from the cornea of the subject's eye; and a corneal imaging lens module 12 configured to focus the returning light from the cornea of the subject's eye to form an image of the cornea of the subject's eye with the ring light pattern on the corneal image sensor.

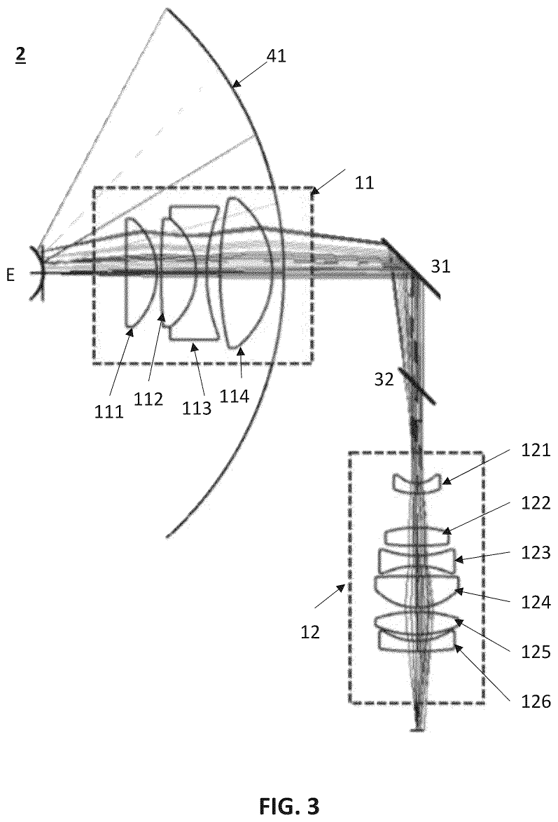

The projector 41 may be configured as a Placido ring light generator to generate a concentric plurality of ring-shaped patterns on to the cornea of the subject's eye. Referring to FIG. 4A, the projector 41 includes a concentric plurality of ring light sources. Each ring light sources includes a ring of LED emitters 411. Essentially, the concentric plurality of ring light sources forms a Placido disk as shown in FIG. 4B to project a plurality of Placido rings on to the cornea. By capturing and analyzing the reflection of the Placido rings by the cornea, the shape and curvature of the any irregularities in the corneal shape, such as steep or flat areas, distort the reflected pattern.

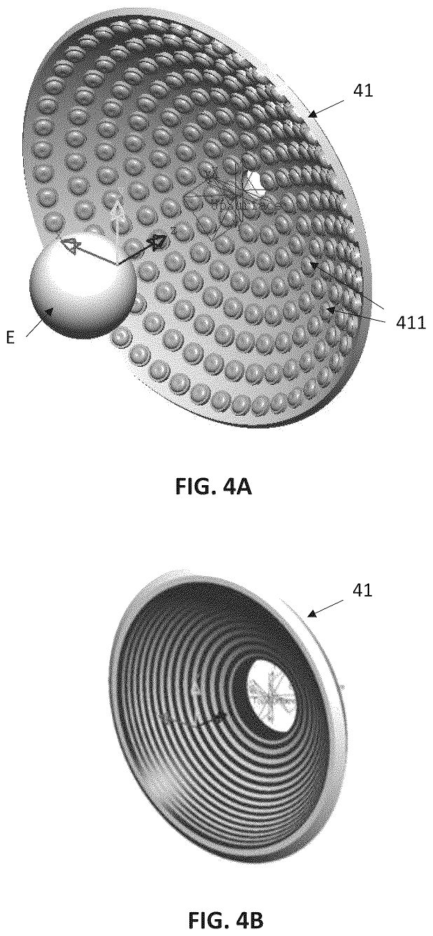

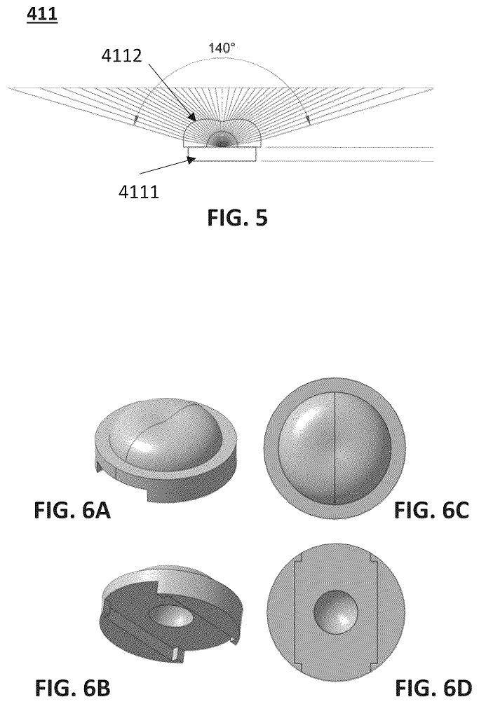

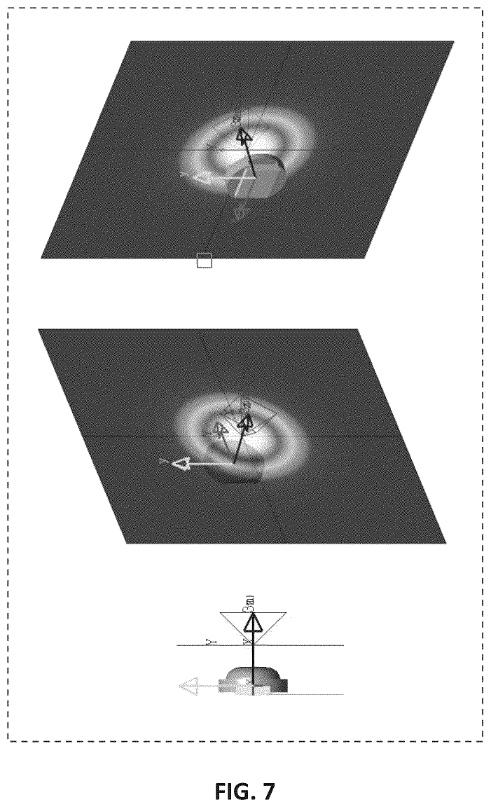

Referring to FIG. 5, each LED emitter 411 includes a near infrared LED 4111 and a primary lens 4112 disposed on top of the LED. FIGS. 6A to 6D show front isometric view, rear isometric views, front view and rear view of the primary lens. FIGS. 7 and 8 show beam profile measurement results of the LED emitter. By implementing the primary lens, the LED emitter can have a beam angle of around 140°; and a beam illumination uniformity (i.e., percentage of variation in the distribution of radiance levels across the beam) greater than 85%.

The corneal image sensor may be selected from a charge-coupled device (CCD) sensor, a complementary metal-oxide-semiconductor (CMOS) sensor, or any other suitable type of image sensors. The corneal image sensor may be configured to perform imaging of the cornea and outputting image signals regarding the cornea of the subject's eye at a predetermined rate to the processing unit. Based on the image signals, the processing unit is configured to measure the curvature of the cornea and then calculate height of the cornea.

In one embodiment, the corneal imaging lens module 12 may include a first lens 121, a second lens 122 positioned next to the first lens 121, a third lens 123 positioned next to the second lens 122, a fourth lens 124 positioned next to the third lens 123, a fifth lens 125 positioned next to the fourth lens 124, and a sixth lens 126 positioned next to the fifth lens 125. The first lens 121 may be a negative meniscus lens. The second lens 122 may be a biconvex lens. The third lens 123 may be a biconcave lens. The fourth lens 124 may be a biconvex lens. The fifth lens 125 may be a biconvex lens. The sixth lens 126 may be a negative meniscus lens. Each of the lenses 121 to 126 may made of glass, plastics or any suitable optical materials.

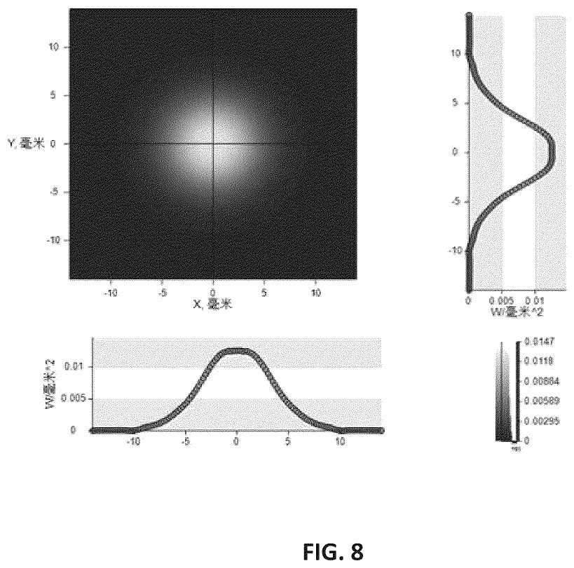

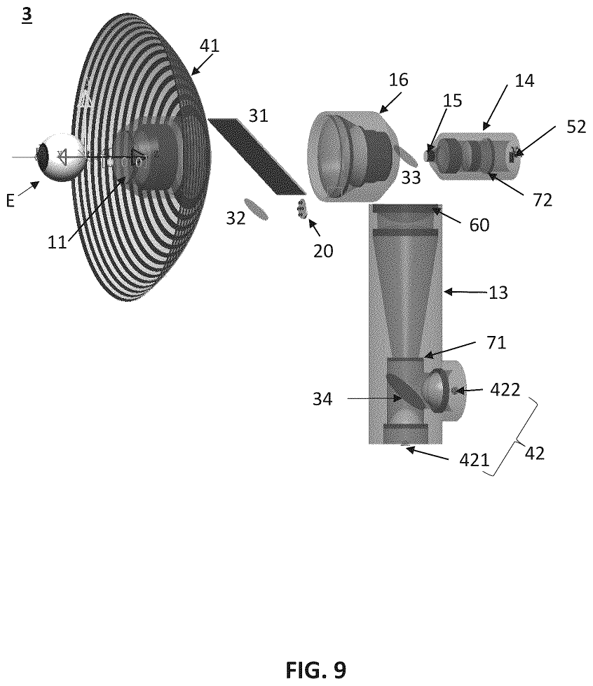

FIG. 9 shows a schematic diagram for the fundus imaging unit 3. FIGS. 10A and 10B show exemplary ray-tracing models for an illumination optical path and an imaging optical path of the fundus imaging unit 3, respectively.

As shown, in addition to the objective lens module 11, the fixture target 20, the first beam splitter 31 and the second beam splitter 32, the fundus imaging unit 3 further includes: a fundus illumination light generator 42 configured to generate a fundus imaging illumination light for capturing image of the fundus; a fundus illumination lens module 13 configured to focus the fundus imaging illumination light; a fundus image sensor 52 configured to detect returning light reflected from the fundus of the subject's eye; a fundus imaging zoom lens module 14 configured to focus the returning light from the fundus of the subject's eye to form an image of the fundus of the subject's eye on the fundus image sensor 52; and a third beam splitter 33 positioned between an intersection point of the imaging optical path and the autofocusing optical path of the fundus imaging unit 3 and configured for diverting lights between the fundus illumination light generator 42 and the subject's eye as well as between the fundus image sensor 52 and the subject's eye.

In one embodiment, the fundus illumination lens module 13 may include a first lens 131 and a second lens 132 positioned next to the first lens 131. The first lens 131 may be a plano-convex lens. The second lens 132 may be a plano-convex lens. Each of the lenses 131 to 132 may made of glass, plastics or any suitable optical materials.

The fundus image sensor 52 may be selected from a charge-coupled device (CCD) sensor, a complementary metal-oxide-semiconductor (CMOS) sensor, or any other suitable type of image sensors. The fundus image sensor 52 may be configured to perform imaging of the fundus and outputting image signals regarding the fundus of the subject's eye at a predetermined rate to the processing unit.

In one embodiment, the fundus imaging zoom lens module 14 may include a first lens 141, a second lens 142 positioned next to the first lens 141, a third lens 143 positioned next to the second lens 142 and a fourth lens 144 positioned next to the third lens 143. The first and second lenses 141 and 142 may form a doublet lens. The third and fourth lenses 143 and 144 may form a doublet lens. The first lens 141 may be a biconcave lens. The second lens 142 may be a biconvex lens. The third lens 143 may be a negative meniscus lens. The fourth lens 144 may be a plano-convex lens. Each of the lenses 141 to 144 may made of glass, plastics or any suitable optical materials.

The fundus imaging zoom lens module 14 may further include an autofocusing lens 15 positioned between the third beam splitter 33 and the first lens 141, and configured for adjusting an effective focal length of the fundus imaging zoom lens module 14. In one embodiment, the autofocusing lens 15 may be a liquid lens connected to the controlling unit. The controlling unit is configured to analyze sharpness of an image of the fundus captured by the fundus image sensor 52 under illumination of the autofocusing illumination light and generate an electrical signal to control the focal length of the liquid lens until a sharp image is obtained.

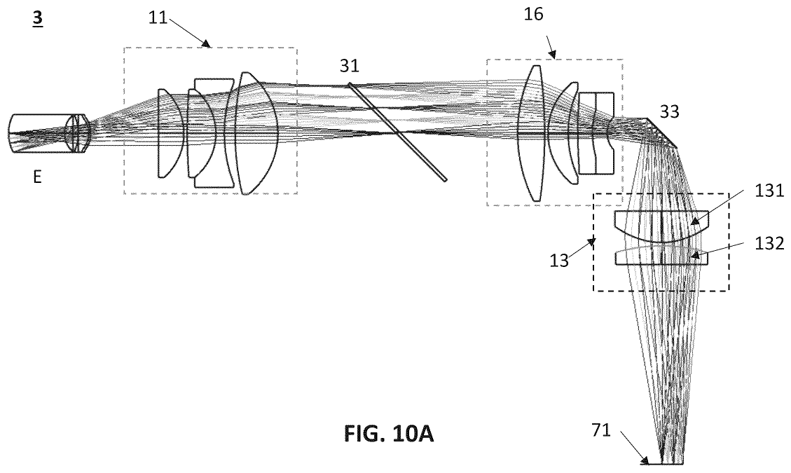

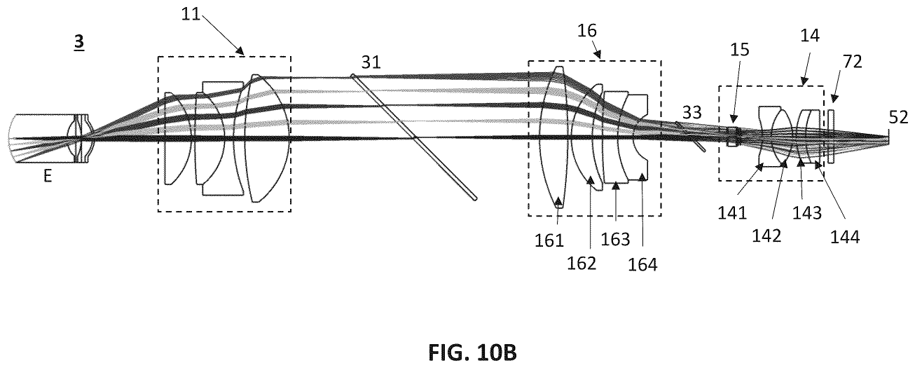

In some embodiments, the fundus illumination light generator 42 may include an imaging illumination light source 421 configured to generate a white ring light as the imaging illumination light; and an autofocusing illumination light source 422 configured to generate a near infrared ring light as the autofocusing illumination light. FIG. 11 shows an exemplary design for the illumination light source 421/422; FIGS. 12A and 12B show front view and rear view of the illumination light source 421/422 respectively. As shown, the illumination light source 421/422 may include a ring of LED emitters 4211/4221 and a PCB 4212/4222 for mounting the LED emitters 4211/4221 and provide electrical connection between the LED emitter 4211/4221 to the controlling unit.

In some embodiments, the imaging and autofocusing illumination light sources 421 and 422 may be set orthogonally. The fundus illumination light generator 42 may further include a fourth beam splitter 34 positioned at an intersection point of optical axes of the imaging illumination light source 421 and autofocusing illumination light source 422 and configured to combine and direct the imaging illumination light and the autofocusing illumination light to the fundus illumination lens module 13.

The fundus imaging unit 3 may further include a relay lens module 16 positioned between the objective lens module 11 and the fundus imaging zoom lens module 14, configured for extending the imaging optical path and illumination optical path of the fundus imaging unit 3.

In one embodiment, the relay lens module 16 may include a first lens 161, a second lens 162 positioned next to the first lens 161, a third lens 163 positioned next to the second lens 162 and a fourth lens 164 positioned next to the third lens 163. The third and fourth lenses 163 and 164 may form a doublet lens. The first lens 161 may be a biconvex lens. The second lens 162 may be a positive meniscus lens. The third lens 163 may be a negative meniscus lens. The fourth lens 164 may be a negative meniscus lens. The third and the fourth lenses 163 and 164 may form a doublet lens. Each of the lenses 161 to 164 may made of glass, plastics or any suitable optical materials.

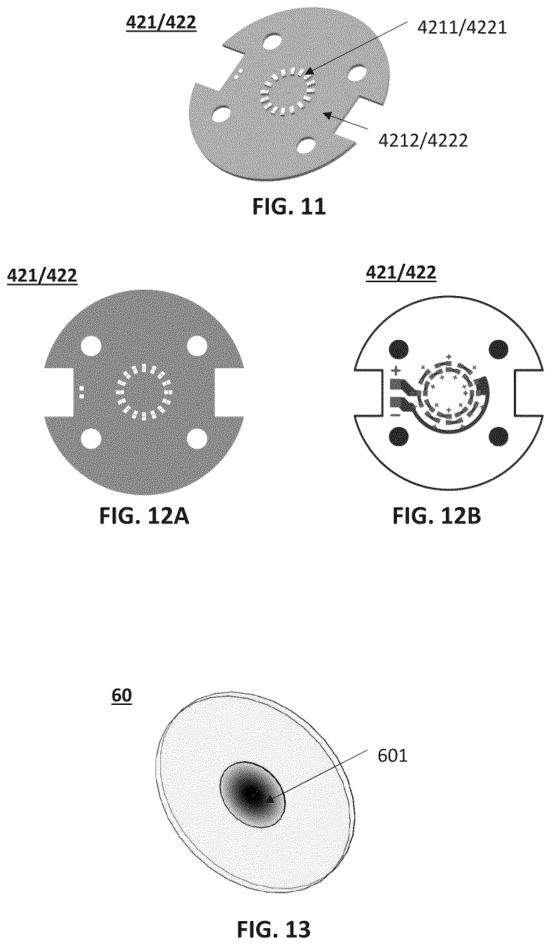

The fundus imaging unit 3 may further include a gradient light blocking filter 60 positioned on an optical path between the fundus illumination lens module 13 and the fundus imaging zoom lens module 14; a first polarization plate 71 positioned between the fundus illumination light generator 42 and the fundus illumination lens module 13; and a second polarization plate 72 positioned between the fundus imaging zoom lens module 14 and the fundus image sensor 52. The polarization directions of the first and second polarization plates 71 and 72 are set orthogonally. The gradient light blocking filter 60, and the first and second polarization plates 71 and 72 form a stray light elimination module configured to remove stray lights generated in the fundus imaging unit 3, such as stray lights from the cornea, the objective lens module 11, the fundus illumination lens module 13 and the relay lens module 16. FIG. 13 shows an exemplary design for the gradient light blocking filter 60. As shown, the gradient light blocking filter 60 may have a blocking area 601 with light transmission rate being lowest at the center and gradually increasing from center to periphery.

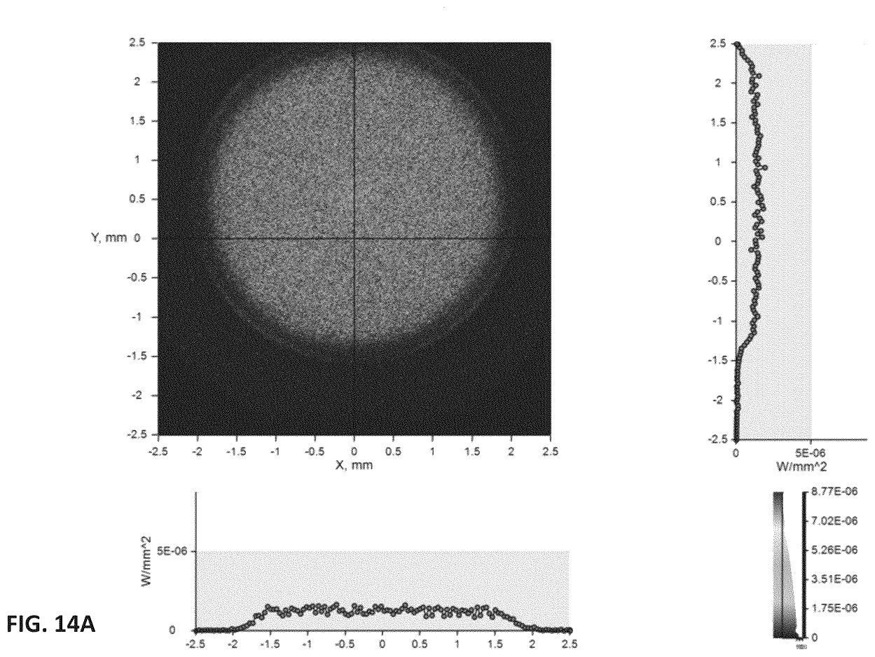

FIGS. 14A and 14B show simulation of fundus image obtained with the fundus imaging unit in accordance with the present invention and a comparative design which using a single LED without stray light elimination unit. As shown, the present invention can provide a more uniform illumination profile and avoid any unwanted light spot as would occur with the comparative design.

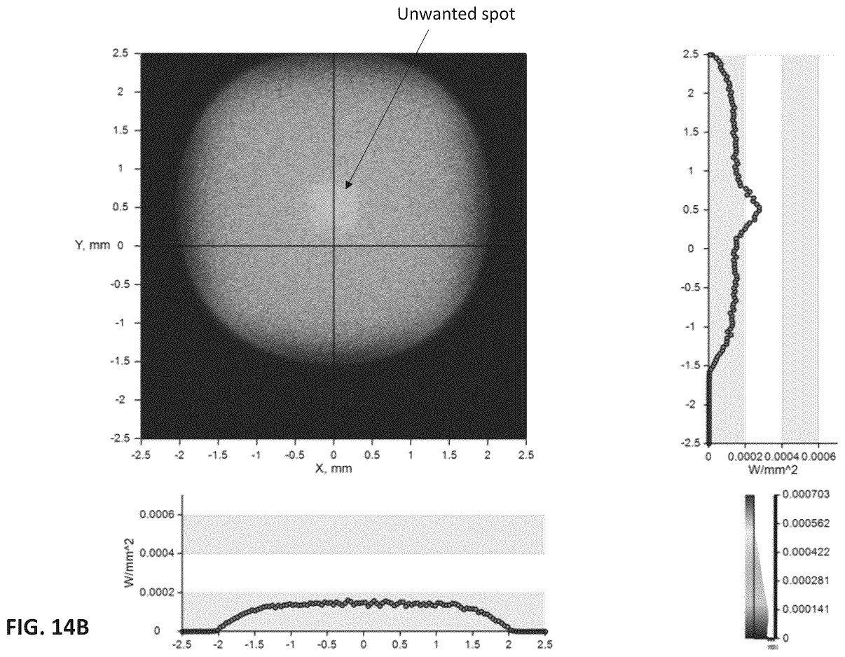

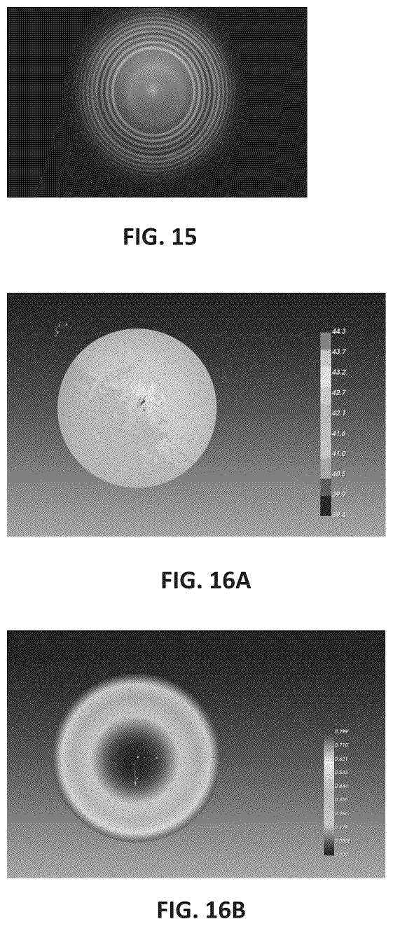

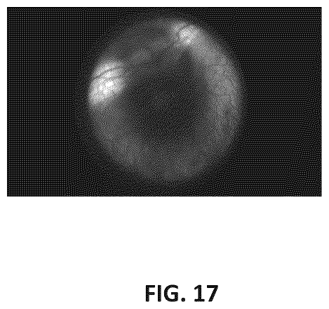

FIG. 15 shows a corneal image obtained by the ophthalmic apparatus provided by the present invention. FIGS. 16A and 16B show a 3D model of corneal curvature and corneal height reconstructed from the corneal image data respectively. FIG. 17 shows a fundus image obtained by the ophthalmic apparatus provided by the present invention.

Based on the calibrated optical path length difference, autofocusing adjustment values and average corneal curvature value, the axial length of the subject's eye can be estimated. In particular, the axial length can be estimated by the formula:

$${L}_{A} = 24 - ( {P}_{R} + {R}_{C C} - 43 + a ( 3.6 - {D}_{A C} ) ) / a , \qquad ( 1 )$$

where L_{A} represents the axial length of the subject's eye, D_{AC} represents the anterior chamber depth, and P_{R} represents the refractive power of the subject's eye and R_{CC} represent the corneal curvature radius in unit of diopter and a is an empirical constant.

By adjusting the driving current I_{liquid} for controlling the liquid lens curvature, the relationship between the refractive power P_{R} and liquid lens driving current I_{liquid} is calibrated, allowing the refractive power P_{R} of the subject's eye can be used as an intermediate quantity for measuring axial length of the subject's eye.

By performing calibration through using a standard artificial human eye model as a calibration sample and changing the refractive power of human eye model together with the known axial length of eye model, the values for the anterior chamber depth D_{AC} and constant a are found to be 3.4 mm and 2.25D-respectively. Eq. (1) can then be simplified as:

$${L}_{A} = 24 - ( {P}_{R} + {R}_{C C} - 43 + 0.45 ) / 2.25 . \qquad ( 2 )$$

Since P_{R} is the calibrated parameter and R_{CC} can be derived from the topography image of the corneal measurement unit, axial length L_{A} of the subject's eye is obtained as a final output.

While the present disclosure has been described and illustrated with reference to specific embodiments thereof, these descriptions and illustrations are not limiting. The illustrations may not necessarily be drawn to scale. There may be distinctions between the artistic renditions in the present disclosure and the actual apparatus due to manufacturing processes and tolerances. There may be other embodiments of the present disclosure which are not specifically illustrated. Modifications may be made to adapt a particular situation, material, composition of matter, method, or process to the objective and scope of the present disclosure. All such modifications are intended to be within the scope of the claims appended hereto. While the methods disclosed herein have been described with reference to particular operations performed in a particular order, it will be understood that these operations may be combined, sub-divided, or re-ordered to form an equivalent method without departing from the teachings of the present disclosure. Accordingly, unless specifically indicated herein, the order and grouping of the operations are not limitations.

What is claimed is:

1. An ophthalmic apparatus, comprising:
an objective lens module configured for magnifying images of a subject's eye;
a fixture target configured for stabilizing the subject's eye to a fixation position;
a corneal topography measurement unit for measuring geometric parameters of a cornea of the subject's eye;
a fundus imaging unit for imaging a fundus of the subject's eye;
a controller configured for controlling the corneal topography measurement unit and the fundus imaging unit to acquire ophthalmic data of the subject's eye;
a processor configured for performing predetermined processing on the acquired ophthalmic data;
a first beam splitter configured for separating optical paths from the objective lens module to the corneal topography measuring unit and the fundus imaging unit or combining optical paths to the objective lens module from the corneal topography measuring unit and the fundus imaging unit respectively; and
a second beam splitter configured for diverting an optical beam from the fixture target to the object's eye;
wherein the objective lens module, the fixture target, the first beam splitter and the second beam splitter are shared by the corneal topography measurement unit, the fundus imaging unit.

2. The ophthalmic apparatus according to claim 1, wherein the corneal topography measurement unit includes:
a projector configured to project a ring light pattern onto the cornea of the subject's eye;
a corneal image sensor configured to detect returning light of the ring light pattern reflected from the cornea of the subject's eye; and
a corneal imaging lens module configured to focus the returning light from the cornea of the subject's eye to form the image of the cornea of the subject's eye with the ring light pattern on the corneal image sensor.

3. The ophthalmic apparatus according to claim 2, wherein the projector is configured as a Placido ring light generator to generate a concentric plurality of ring-shaped patterns on to the cornea of the subject's eye.

4. The ophthalmic apparatus according to claim 3, wherein the Placido ring light generator includes a concentric plurality of Placido ring light sources.

5. The ophthalmic apparatus according to claim 4, wherein each Placido ring light source includes a ring of LED emitters; and each LED emitter includes a LED and a primary lens disposed on top of the LED.

6. The ophthalmic apparatus according to claim 1, wherein the fundus imaging unit includes:
a fundus illumination light generator configured to generate an imaging illumination light for capturing image of the fundus;
a fundus illumination lens module configured to focus the primary fundus illumination light;
a fundus image sensor configured to detect returning light reflected from the fundus of the subject's eye; and
a fundus imaging zoom lens module configured to focus the returning light from the fundus of the subject's eye to form the image of the fundus of the subject's eye on the fundus image sensor.

7. The ophthalmic apparatus according to claim 6, wherein
the fundus imaging zoom lens module includes an autofocusing lens for adjusting a focal length of the fundus imaging zoom lens module; and
the fundus illumination light generator is further configured to generate an autofocusing illumination light for adjusting the focal length of the fundus imaging zoom lens module.

8. The ophthalmic apparatus according to claim 7, wherein the fundus illumination light generator includes:
an imaging illumination light source configured to generate a white ring light; and
an autofocusing illumination light source configured to generate a near infrared ring light.

9. The ophthalmic apparatus according to claim 6, wherein the fundus imaging unit further includes a relay lens module positioned between the objective lens module and the fundus imaging zoom lens module, and configured for extending the imaging optical path and illumination optical path of the fundus imaging unit.

10. The ophthalmic apparatus according to claim 1, wherein the fundus imaging unit further includes:
a gradient light blocking filter positioned on an optical path between the fundus illumination lens module and the fundus imaging zoom lens module;
a first polarization plate positioned between the fundus illumination light generator and the fundus illumination lens module; and
a second polarization plate positioned between the fundus imaging zoom lens module and the fundus image sensor; and
wherein the first and second polarization plates are set orthogonally; and
the gradient light blocking filter, the first polarization plate and the second polarization plate form a stray light elimination module configured to eliminate reflection of stray light in the fundus imaging unit.
